(19) United States
(12) Patent Application Publication
(10) Pub. No.: US 2004/0175432 A1
(43) Pub. Date: Sep. 9, 2004

(54) Metallic comb incorporating copper salts

(76) Inventor: Victor Alejandro ESCUDERO GACITUA, Santiago (CL)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/382,463

(22) Filed: Mar. 6, 2003

(51) Int. Cl. A61K 33/38; A61K 33/34; A45D 24/00

(52) U.S. Cl. 424/618; 424/630; 132/219

(57) ABSTRACT

Metallic comb for dosing copper salts to the user. The comb can be pure copper metal or an alloy which can include at least one of copper, antimony, nickel, silver and arsenic. Normal use of the comb provides a residuum of copper salts to be deposited in the hair.

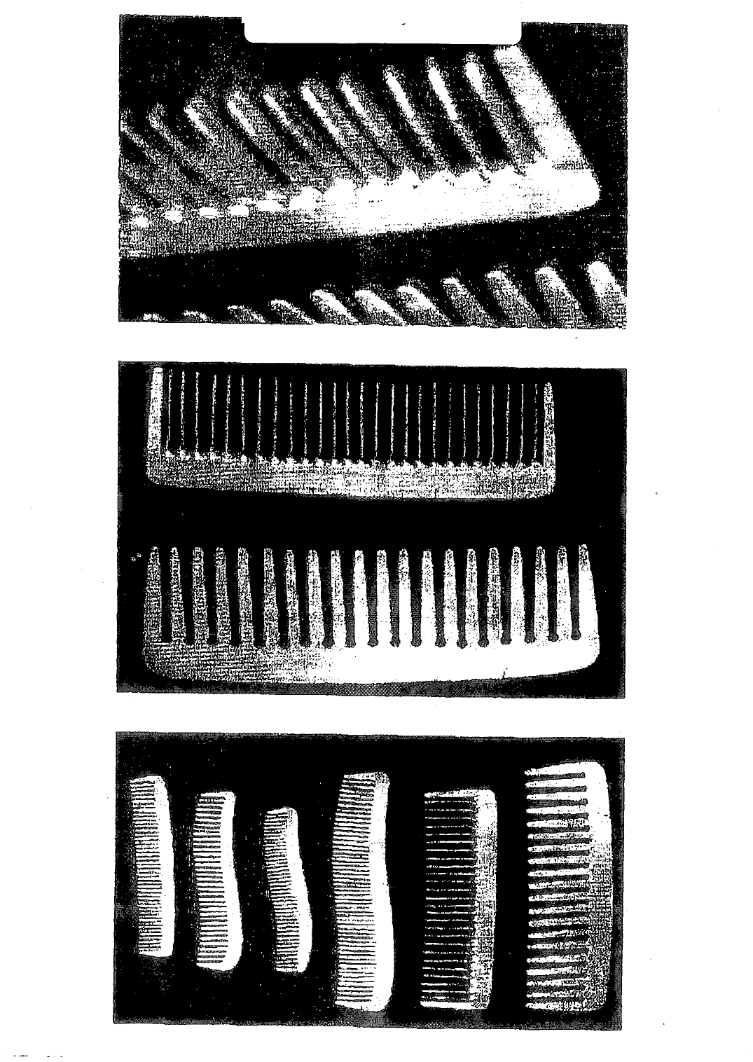

### EXPLANATORY ACCOUNT

The current invention refers to the manufacturing of a device which can incorporate copper salts to the hair or the scalp to prevent the development off microorganisms and the elimination of plagues through a metallic hair comb with a wearing away surface which has a composition of 98% of pure copper and 2% of different metals.

### BACKGROUND OF THE INVENTION

In the present technique state, it exists a number of different objects to eliminate different types of simple organisms in the hair which act by hauling, electrocution, caught by fine hair combs etc.

The patent request 00255-1991 describes a manual devise to comb the hair, which destroys louses, fleas and other plagues through electrocution.

The U.S. Pat. No. 4,729,147 describes an exterminator of dog fleas and a devise to clean pets.

Another known way to eliminate these types of organisms is the application of chemical products through the well known shampoos, which are nothing else but soaps with toxic admixtures which are very good for eliminating the organisms but no to prevent them.

These are the registered patents cases that show the state of the technique in which exists no manual and simple object to prevent the problems of pediculosis, fungus, bacteria, etc.

### DESCRIPTION OF THE INVENTION

The present invention deals with a metal indented devise (comb like), which gives the hair and scalp salts and copper oxides in both their ionic states. The salts are given by the superficial liberation of the natural oxidation of copper being used as comb.

Copper in its natural state remains protected through a fine film of oxide and according to the valence with which it acts, it will rust as cupric oxide of a dark brown color or as cuprous oxide of an ocher color to purple red and, in contact with humidity and air contamination it develops salts such as chlorides and sulfates of a greenish color. All which has a certain grad of toxicity, depending on its concentration.

Actually different types of copper salts are manufactured in the industry to fight a variety of simple or complex microorganisms such as plagues, fungus, bacteria, etc. which cause of sicknesses and infections.

Being copper salts a compound of so many different applications, the cuprous oxide is found in the making of anti-incrusting or anti-fouling pigments with which the underwater part of ships are painted to avoid the development of microorganisms colonies, seaweed and Mollusca which produces a greater resistance by friction, to advance with the resulting from a higher consume of fuel, besides the damage caused to the steel structure. It is also applied to structures in wharf, buoy, fishing nets, pillars, etc. (according to the Pesticide Ordinance Cap.133 regulations administrated by the Conservation an Agricultural Department of the United States of America).

In pharmacology, in Chile Agua de Alibur, which is a dissolved copper sulfate solution used to disinfect, that is applied directly on the wound without producing any shin irritation or damage.

In agriculture the Copper Oxychloride as fungicide for citric trees, fruit trees, vegetables, etc. Copper sulfate is used too to impregnate woods, pillars, etc. to avoid moths damages or rot as a result from fungus.

In cattle raising copper sulfate is used to disinfect fungus from cattle and goats hoofs, etc. It is also used to disinfect the water that animals drink, killing in this way the small snails which transmits liver fluke.

In laboratory experiments comparative evaluations have been made of the development of bacteria in drink water in contact with different types of plumbing tubes. It has been demonstrated that “of all the materials used copper, copper had the greater success in limiting the development and inhibited the growth of bacteria”, according to investigations made by the Center of Applied Microbiology & Research IAC in their reports of projects

Copper is no risk for human health. Through history copper has been used to make coins, jewels, kitchen utensils, etc. which are used in direct contact with human skin.

The casual ingestion of micro-particles of copper through the handling of the comb is no risk for health, on the contrary it has been proved that it is commendable to take 2 to 3 milligrams of copper in the daily diet. This dose is insignificant, it prevents us cardiovascular sicknesses, osteoporosis, cancer, etc. and it is vital to maintain the skin and blood vases flexible and elastic. The chronic deficiency of copper in our organism would provoke anemia, leukopenia and osseous demineralization (according to documents of Copper—fact and information (WWW.go-symmentry.com/copper)).

In medicine, the use of copper mid its derivatives such as disinfectants fungicides ercides, etc. is recognized according to the document Copper Benefits in Health by Dr. Manuel Olivaes G. MD Director of the Laboratorios Microminerales del Instituto de Tecnologia de los Alimentos de la Universidad de Chile licromineral Laboratory of The Food Technology Institute of the University of Chile).

Given the variety and successful appliances which derive from copper has and, in face of the problems caused by the apparition of plagues in the hair like louse eggs and louse, mites fungus, etc. the purpose of the current invention is to improve the health and vitality of hair and scalp; adding a very small quantity of salts and copper oxide through the daily and reiterative use of this object used as a comb which wears away the rusted surface on the hair by the friction which generates its use.

The form of an indented comb has the purpose to give a greater contact surface for rusting as well as for passing over this rust to the hair which would remain with a slight an imperceptible metal smell, enough to generate a toxic milieu for the nesting, egg laying and proliferation of these simple organisms as well for the development of fungus and spores.

The application of these copper salts on the hair, through the superficial away wearing of the metal comb during its use, allows to give away a natural dosage due to the sole interaction of this device used as a comb. It would also act as a preventive treatment. The small dose obtained by the interaction of the metal comb with the hair makes this invention as a very reliable and economic dispenser for massive use. Furthermore it is very reliable because once its surface is cleaned by the hairs, it requires some time to re-rust its self

These attributes of the copper salts on the hair would act as:

Bactericides: Would avoid the development of bacteria and infections, etc., which in some way causes baldness and bad smell.

Fungicide: It would destroy the spores of fungus widespread on the scalp which generates the bad smells, surface and deep desquamation which causes loss of hair by the weakening of the roots.

Germicide: These attributes of copper salts given away by the copper comb would generate a toxic milieu for the nesting of simple organisms like louse eggs, louse, mites, etc. avoiding, in this way, its reproduction and development

Statics: It discharges the static electricity generated by the friction of the comb thus avoiding the well known hair flowering or hair death due to successive electric discharges on the hair tips.

The basic composition of the copper salts dosage comb is the following:

| | | |
|---|---|---|
| Metal copper | pure 100% | 99.810% |
| Metal Antimony | pure 100% | 0.088% |
| Metal Nickel | pure 100% | 0.080% |
| Metal Silver | pure 100% | 0.020% |
| Metal Arsenic | pure 100% | 0.008% |

With this composition forms an alloy which is specific for the dosage of the salts which allows to create an adverse surrounding for the mentioned organisms.

1. Dosage comb of copper salts CHARACTERIZED because the material with which it is made of includes any type of metal.

2. Dosage comb of copper salts CHARACTERIZED according to claim N^{o}1 because the metal can be pure or an alloy.

3. Dosage comb of copper salts CHARACTERIZED according to claim N^{o} 2 because the pure metal is copper.

4. Dosage comb of copper salts CHARACTERIZED according to claim N^{o} 2 because the alloy is made of various metals.

5. Dosage comb of copper salts CHARACTERIZED according to claim N^{o} 4 because the alloy of metals is made of the following elements: Copper, Antimony, Nickel, Silver, Arsenic.

6. Dosage comb of copper salts CHARACTERIZED according to claim N^{o} 5 because the metal alloy is made of:
1-99.99% or metal Copper→preferably 99.81%
0,0001-0.19% of metal Antimony→preferably 0.088%

7. Dosage comb of copper salts CHARACTERIZED according to claim N^{o} 5 because the metal alloy is made of:
1-993.99% of metal Copper→preferably 99.81%
0.0001- 0.19% metal Nickel→preferably 0.080%

8. Dosage cormb of copper salts CHARACTERTZED according to claim N^{o} 5 because the metal alloy is made of
1-99.99% of metal Copper→preferably 99.81%
0.0001-0.19% of metal Silver→preferably 0.020%

9. Dosage comb of copper salts CHARACTERIZED according to claim N^{o} 5 because the metal alloy is made of:
1-99.99% of metal Copper→preferably 99.81%
0.0001-0.19% of metal Arsenic→preferably 0.008%

10. Dosage comb of copper salts CHARACTERV/ED according to claim N^{o} 1 because its design is basicaly a rectangular body which has a set of teeth or metal points which come out of the first third of the body and prolongues through the rest of the two thirds of the mentioned body

11. Dosage comb of copper salts CHARACTERIZED according to claim N^{o} 10 because it includes any type of hair fastener made of the type of materials detailed on the above mentioned claims.

12. Dosage comb of copper salts CHARACTERIZED according to claim N^{o} 10 because it includes any type of hair fastening band made of the materials detailed on the above mentioned replevys.
